(19) United States
(12) Patent Application Publication
(10) Pub. No.: US 2020/0297241 A1
(43) Pub. Date: Sep. 24, 2020

(54) WEARABLE COMPUTING DEVICES FOR ACQUIRING ATHLETIC MOVEMENT DATA, AND SYSTEMS AND METHODS RELATING THERETO

(71) Applicant: SPARTA SOFTWARE CORPORATION, Menlo Park, CA (US)

(72) Inventor: Phillip Patrick Wagner, Menlo Park, CA (US)

(21) Appl. No.: 16/837,218

(22) Filed: Apr. 1, 2020

Related U.S. Application Data
(63) Continuation of application No. PCT/US18/55163, filed on Oct. 10, 2018.
(60) Provisional application No. 62/570,427, filed on Oct. 10, 2017.

(51) Int. Cl. A61B 5/103; G16H 20/30; G16H 40/67; A61B 5/00

(52) U.S. Cl. CPC A61B 5/1038; G16H 20/30; G16H 40/67; A61B 5/6807; A61B 5/6832; A61B 5/0022; A61B 2503/10; A61B 2562/0261; A61B 5/002

(57) ABSTRACT

Wearable computing devices adapted to be worn on the feet are provided for acquiring, validating and analyzing athletic movement data. Generally, the wearable computing devices can include one or more processors, a memory, and one or more sensors for sensing certain characteristics of an athletic movement. A local computing device is provided for receiving data from the wearable computing device that is indicative of the characteristics of the athletic movement, extracting selected portions from the data, and transmitting the selected portions to a remote server system. The remote server system can be configured to store, aggregate and update a data in a database, and generate one or more normalized values and/or interventions associated with the athletic movement. The normalized values may indicate to the user a susceptibility to injury, progression towards return to play or propensity for success with respect to a sport.

### CROSS-REFERENCE TO RELATED APPLICATIONS

The subject application is a continuation of PCT Patent Application No. PCT/US18/55163, filed Oct. 10, 2018, which claims priority to U.S. Provisional Patent Application No. 62/570,427, filed on Oct. 10, 2017, both of which are incorporated by reference herein in their entirety for all purposes.

### FIELD

The subject matter described herein relates generally to wearable computing devices for acquiring and validating athletic movement data, and systems and methods relating thereto. In particular, sensor data for a user performing an athletic movement is captured by a wearable computing device comprising one or more sensors. The sensor data can be validated, and selected portions can be extracted and normalized based at least in part on population data. The normalized values may indicate the user's susceptibility to injury, progression towards return to play or suitability for a particular sport.

### BACKGROUND

Advances in kinesiology and sensor technology have enabled researchers to capture and study vast amounts of data regarding the human body during athletic movement. Some researchers, for example, have utilized electromyography (“EMG”) for detecting and recording electrical activity produced by skeletal muscles during a golf swing. Other researchers have employed near-infrared spectroscopy (“NIRS”) to measure and monitor oxygenation in muscle and other tissues during cycling exercises.

Despite an abundance and diversity of data, however, a significant challenge remains in translating the data into actionable results. For example, according to a five-year study of National Football League's (“NFL”) Combine, no consistent statistical relationship could be found between results of the highly-technical Combine tests and the actual performance of professional football players. Similarly, in another study of basketball players, researchers were unable to identify any meaningful patterns in the collected data with respect to the players' injury resilience.

By contrast, some studies using force plates to acquire sensor data associated with vertical jumps and other athletic movements have shown promise. More specifically, some studies have shown that the eccentric rate of force development, which can be derived from data acquired by a force plate, can be associated with athletic performance. Unfortunately, force plates can be expensive, bulky, obtrusive, and relatively immobile. Moreover, force plates, which are typically installed in a “controlled environment,” such as a laboratory or training facility, do not adequately simulate the playing environment of the athlete. Additionally, little effort has been made to measure the ground reaction forces generated by the athlete while simultaneously acquiring other types of sensor data generated during an athletic movement.

Therefore, there is a need for improved systems, devices and methods for acquiring athletic movement data, such as data associated with ground reaction forces. There is also a need for validating and analyzing said data, and in particular, for the purpose of determining an athlete's susceptibility to injury, progression towards return to play or suitability for a particular sport.

### SUMMARY

Provided herein are example embodiments of wearable computing devices for acquiring athletic movement data, as well as systems and methods relating thereto. Generally, one or more wearable computing devices are provided, wherein the wearable computing device is configured to be worn on a user's foot and comprises one or more sensors that are adapted to sense various characteristics of various athletic movements. In some embodiments, these characteristics may include, for example, ground reaction forces generated during an athletic movement. In other embodiments, these characteristics may include acceleration forces. The wearable computing device may include one or more processors and a memory coupled thereto. The memory can store instructions that, when executed by the one or more processors, cause the one or more processors to perform various method steps for acquiring, validating and storing athletic movement data. The wearable computing device can also include a wireless communications module configured to transmit the stored sensor data to a local computing device for further processing.

A local computing device may be provided for receiving the stored sensor data, and can comprise one or more processors and a memory coupled thereto. The memory of the local computing device can store instructions that, when executed by the one or more processors, cause the one or more processors to perform various method steps for processing the stored sensor data.

For example, in some embodiments, the memory of the local computing device can store instructions that cause the processors to extract selected portions from the stored sensor data, normalize the selected portions of sensor data, and determine and display an intervention based on the normalized selected portions of sensor data. The local computing device can also include a wireless communication module for communicating with one or more wearable computing devices, and a network interface module for communicating with a remote server system.

A remote server system may also be provided for receiving and storing the processed sensor data and can be configured for transmitting to the local computing device one or more normalized values correlating to the processed sensor data associated with the one or more athletic movements. The normalized values can include T-scores, which are normalized by various factors, such as by body weight, by gender, by preferred sport or by preferred position within a sport. In addition, the remote server system may include a database comprising stored processed sensor data indicative of characteristics of various athletic movements for a population of athletes. In this regard, and as described in further detail below, the normalized values may provide a variety of indicators and interventions to an athlete such as, for example, susceptibility to injury, progression towards return to play, or suitability for a particular sport, to name a few.

To ensure the integrity of the acquired sensor data, data validation methods are also provided. For example, in some embodiments, prior to the user performing an athletic movement, the wearable computing device can measure a weight of the user and compare it to a stored reference weight. If a weight mismatch is detected, e.g., if the measured weight is inaccurate or the user has misidentified herself, then the wearable computing device can refrain from acquiring further sensor data and expending resources (e.g., processor, memory, power). In other embodiments, one or more predetermined thresholds are monitored during the athletic movement which may detect, for example, a user performing an athletic movement with insufficient force, insufficient velocity, or insufficient acceleration. In response to the predetermined thresholds not being exceeded, the wearable computing device can refrain from storing sensor data. These and other data validation methods are described in further detail below.

These embodiments and others described herein are improvements in the fields of computer-assisted biomechanics and, in particular, in the area of computer-based kinetic and kinematic analysis. Other systems, devices, methods, features, and advantages of the subject matter described herein will be apparent to one with skill in the art upon examination of the following figures and detailed description. The various configurations of these systems, devices, methods, features, and advantages are described by way of the embodiments which are only examples. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an example embodiment of a system for acquiring athletic movement data comprising one or more wearable computing devices.

FIG. 2 is a block diagram of an example embodiment of a wearable computing device.

FIG. 3 is a block diagram of an example embodiment of a local computing device.

FIG. 4 is a block diagram of an example embodiment of a remote server system.

FIGS. 5A and 5B are perspective, exploded views of example embodiments of wearable computing devices.

FIGS. 6A to 6D are top sectional views of example embodiments of wearable computing devices.

FIGS. 7A and 7B are side, exploded views of example embodiments of wearable computing devices.

FIG. 8A is a back view of one aspect of an embodiment of a wearable computing device, shown in three stages.

FIG. 8B is a top view of an example embodiment of a wearable computing device.

FIG. 9 is a flow chart diagram depicting an example embodiment method for acquiring athletic movement data.

FIG. 10 is a flow chart diagram depicting another example embodiment method for acquiring athletic movement data.

FIG. 11 is a flow chart diagram depicting an example embodiment method for acquiring athletic movement data using multiple wearable computing devices.

FIG. 12 is a flow chart diagram depicting an example embodiment method for acquiring athletic movement data relating to pronation-supination of a user's foot.

FIG. 13 is a flow chart diagram depicting an example embodiment method for acquiring athletic movement data relating to foot rotation.

FIG. 14 is a block diagram of another example embodiment of a wearable computing device.

FIG. 15 is a flow chart diagram depicting another example embodiment method for acquiring movement data.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure comprise wearable computing devices for acquiring athletic movement data, and systems and methods relating thereto. Accordingly, many embodiments may include one or more wearable computing devices comprising one or more sensors, wherein the one or more sensor devices are configured to sense various characteristics of an athletic movement performed by a user. In addition, many embodiments may also include a local computing system configured to receive stored sensor data from the wearable computing device, and a remote server system which may include, or be communicatively coupled with, a database configured to store sensor data associated with various athletic movements for a population of athletes.

In some embodiments, for example, a first wearable computing device is provided, wherein the wearable computing device is adapted to be worn on a first designated foot of a user. The wearable computing device can include one or more force-measuring sensors, one or more processors coupled to the force-measuring sensors, and a memory (also coupled to the processors) for storing instructions that, when executed by the processors, cause the processors to detect signals generated by the sensors, determine ground-reaction force values from the signals, and in response to the ground-reaction force values exceeding a predetermined threshold, store the ground-reaction force values in the memory of the wearable computing device. In some embodiments, the wearable computing device may also include other types of sensors, such as accelerometers, magnetometers and gyroscopic sensors, to name only a few. In still other embodiments, the wearable computing device may include only one or more accelerometers, magnetometers and/or gyroscopic sensors—without force-measuring sensors.

In other embodiments, a local computing device is provided, wherein the local computing device is configured to receive stored sensor data, e.g., stored ground-reaction force values from one or more wearable computing devices. The local computing device can include one or more processors and a memory for storing instructions that, when executed by the processors, cause the processors to extract selected portions of the received sensor data, normalize the selected portions of the received sensor data, and determine and/or display an intervention associated with the normalized values.

In still other embodiments, a remote server system is provided, wherein the remote server system can include, or be communicatively coupled with, a database comprising stored sensor data associated with various athletic movements for a population of athletes. According to one aspect of these embodiments, the remote server system can be configured to receive selected potions of sensor data from the local computing device, normalize the selected portions of the sensor data, determine an intervention associated with the normalized values, and transmit the normalized values and the intervention to the local computing system for display.

Additionally, the present disclosure may also include systems and methods for validating the data acquired by the one or more sensors of the wearable computing device, and may include, for example, a weight validation process, a minimum force process, a minimum velocity process, and a minimum acceleration process, each of which is described in further detail below. Other sensor data validation processes are described in U.S. Patent Application Ser. No. 62/528,866, which is incorporated by reference in its entirety for all purposes. Further, the embodiments disclosed herein may include local computing devices, each of which may be in communication with a remote server system that is location-independent, i.e., cloud-based. Those of skill in the art will also appreciate that the embodiments disclosed herein may also include local computing devices, each of which may be in communication with a remote server system that is located on the same premise and/or local area network as the one or more local computing devices. In these embodiments, the remote server systems, which are located on the same premise and/or local area network as the one or more local computing devices, may also be configured to synchronize a database containing stored sensor data associated with a population of athletes with a database residing on, or coupled with, a centralized remote server system that is location-independent, i.e., cloud-based.

Furthermore, for each and every embodiment of a method disclosed herein, systems and devices capable of performing each of those embodiments are covered within the scope of the present disclosure. For example, embodiments of wearable computing devices, local computing devices, and remote server systems are disclosed, and these devices and systems may each have one or more sensors, analog-to-digital converters, one or more processors, memory for storing instructions, displays, storage devices, communications modules (for wired and/or wireless communications), and/or power sources, that may perform any and all method steps, or facilitate the execution of any and all method steps.

The embodiments of the present disclosure provide for improvements over prior modes in the field of computer-based kinetic and kinematic analysis. These improvements may include, for example, optimization of computer resources, improved data accuracy and improved data integrity, to name only a few. In a number of embodiments, for example, instructions stored in the memory of a wearable computing device (e.g., software or firmware) may cause one or more processors of the wearable computing device to determine whether certain characteristics (or combination of characteristics) derived from signals received by one or more sensors exceed one or more predetermined thresholds. If the thresholds are not met or exceeded, then the sensor data is not stored or processed, thereby conserving significant computing resources of the wearable computing device.

According to another aspect of some embodiments disclosed herein, instructions stored in the memory of a local computing device (e.g., software or firmware) may cause one or more processors of the local computing device to process and extract certain characteristics from sensor data associated with one or more athletic movements received from the wearable computing device, and transmit the processed sensor data to a remote server system. Subsequently, the remote server system receives and stores the processed sensor data, and returns to the local computing device one or more normalized values correlating to the athletic movement. The normalized values may be T-scores, for example, and displayed on the local computing device. The sensor data on the local computing device may be subsequently discarded. Thus, according to one aspect of the embodiments, memory and hard drive space are conserved at the local computing device because sensor data need not be permanently stored. Likewise, the remote server system need only store extracted portions of sensor data, thereby conserving memory, hard drive space and processing power. Thus, computer resources may be significantly conserved both at the local computing device as well as at the remote server system.

The disclosed embodiments also reflect computer-related improvements in data accuracy and integrity. In some embodiments, for example, the remote server system includes, or is communicatively coupled with a database for storing sensor data correlating to a population of athletes. According to one aspect of the disclosed embodiments, the remote server system may be location-independent (i.e., cloud-based), and configured to aggregate sensor data from a plurality of local computing devices, which may be located in a plurality of geographically dispersed areas. The remote server system may also provide normalized values to each local computing system based on the population data contained in the database. The normalized values may also be normalized according to categories, for example, by gender, by body weight, by sport or by position within a sport. By continually aggregating and updating the population data contained within the database by category, the remote server system may be configured to provide customizable, dynamically-generated and accurate values to the user.

According to another aspect of the disclosed embodiments, improvements in data integrity are also provided through data validation processes during the acquisition of the sensor data. As described in further detail below, the data validation processes may include, for example, a weight validation process, a minimum force process, a minimum velocity process, and a minimum acceleration process, each of which is described in further detail below. Other sensor data validation processes are described in U.S. Patent Application Ser. No. 62/528,866, which is incorporated by reference in its entirety for all purposes. Each of these processes, as well as others, are configured to ensure that the acquired sensor data is accurate and correct prior to processing and receiving the extracted sensor data by the remote server system.

The improvements of the present disclosure are necessarily rooted in computer-based systems for the acquisition, validation and analysis of athletic movement data, and are directed to solving a technological challenge that might otherwise not exist but for the existence of computer-based kinetic and kinematic analyses. Additionally, many of the embodiments disclosed herein reflect an inventive concept in the particular arrangement and combination of the devices, components and method steps utilized for acquiring, validating and analyzing athletic movement data. Other features and advantages of the disclosed embodiments are further discussed below.

Before describing these aspects of the embodiments in detail, however, it is first desirable to describe examples of devices that may be present within, for example, a system for acquiring, validating, and analyzing athletic movement data, as well as examples of their operation, all of which may be used with the embodiments described herein.

### Example Embodiment of a System for Acquiring Athletic Movement Data

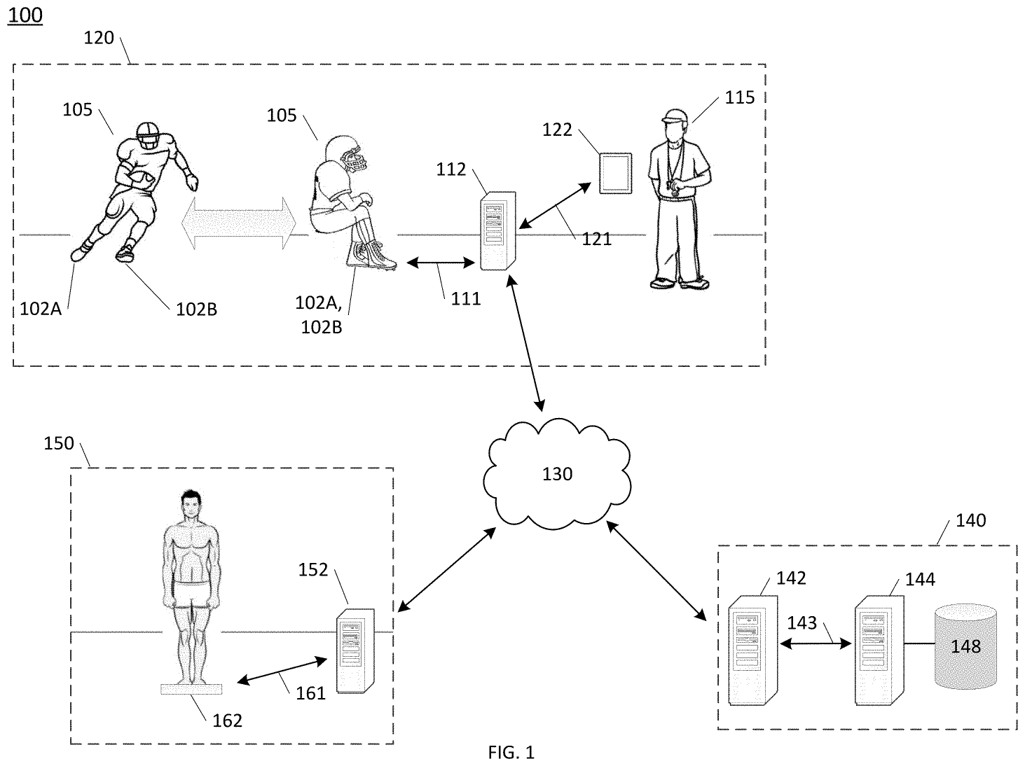

FIG. 1 is a conceptual diagram depicting an example embodiment of a system 100 that includes one or more wearable computing devices for acquiring athletic movement data, and which may be used with the embodiments of the present disclosure. System 100 may include one or more wearable computing devices 102A, 102B adapted to be worn on the feet of user 105. Wearable computing devices 102A, 102B can comprise athletic footwear to be used in a “live” environment 120, such as during a competitive sporting event. In many embodiments, wearable computing devices 102A, 102B can include one or more sensors configured to generate signals in response to athletic movements. According to some embodiments, for example, wearable computing devices 102A, 102B, can include force-measuring sensors adapted to generate signals in response to ground-reaction forces created before, during and after a vertical jump. According to other embodiments, wearable computing devices 102A, 102B, can include accelerometers adapted to generate signals in response to acceleration forces. Wearable computing devices 102A, 102B can also include one or more processors coupled to the sensors, a memory coupled to the processor and a wireless communication module.

As described in further detail below, wearable computing devices 102A, 102B can be adapted to detect signals generated during an athletic movement of user 105, determine a characteristic of the athletic movement (e.g., ground-reaction force value) from the detected signals, and, if certain predetermined thresholds are met or exceeded, store the determined values in memory. Furthermore, in many embodiments, wearable computing devices 102A, 102B can be configured to conserve computing resources when user 105 is at rest. For example, instructions stored in memory of wearable computing devices 102A, 102B can cause the processors to refrain from storing data in memory if a predetermined force, velocity, and/or acceleration threshold is not met, e.g., if user 105 is sitting or walking in environment 120. Furthermore, instructions stored in memory of wearable computing device 102A, 102B can cause processors to wirelessly transmit data, e.g., ground-reaction force values, to a local computing device 112. In some embodiments, these wireless transmissions can occur periodically or continuously, according to a standard wireless networking protocol, such as 802.11x, Bluetooth, Bluetooth Low Energy, Near Field Communication (NFC), UHF, or infrared. Those of skill in the art will recognize that other standard wireless networking protocols are within the scope of the present disclosure. In other embodiments, wearable computing devices 102A, 102B can include a data port to allow for wired transmissions of stored data to local computing device 112. In still other embodiments, wearable computing devices 102A, 102B can include a removable memory device or media (e.g., micro SD memory card) to allow stored data to be transferred to another device, such as local computing device 112.

Referring still to FIG. 1, one or more local computing devices 112 are provided in system 100 for receiving stored data from wearable computing devices 102A, 102B. In many embodiments, data transfer between wearable computing device 102A, 102B and local computing device 112 can occur through a wired or wireless communication link 111, as described earlier with respect to wearable computing devices 102A, 102B. Local computing device 112 can also process and extract selected portions from the received sensor data, and transmit the selected portions over network 130 to remote server system 140. Network 130 may comprise the Internet, a wide area network, a local area network, a metropolitan area network, a virtual private network, a cellular network, or any other type of wired or wireless network. Furthermore, local computing device 112 may be a personal computer, laptop computer, desktop computer, workstation computer, or any other similar computing device.

In some embodiments, as shown in FIG. 1, a mobile computing device 122, such as a tablet computer, laptop, smart phone, or wearable computing device, may also be communicatively coupled to local computing device 112 through a wired or wireless communication link. Mobile computing device 122 may be configured to receive data from local computing device 112 through communication link 121. In some embodiments, mobile computing device 122 can also include a user interface to allow a second user 115 (e.g., coach, trainer, or supervisor) to manage data transfers between wearable computing devices 102A, 102B and local computing device 112. In some alternative embodiments, mobile computing device 122 may be configured to communicate directly with wearable computing devices 102A, 102B through Bluetooth, Bluetooth Low Energy, 802.11x, UHF, NFC or any other standard wireless communications protocol. In some embodiments, mobile computing device 122 may be configured to operate according to a mobile operating system such as Android and/or IOS.

System 100 may also include a remote server system 140 configured to receive data from one or more local computing devices 112, and which may comprise a front-end server 142 for interfacing with said local computing devices 112, and a back-end server 144 that interfaces with both the front-end server 142 and database 148. Remote server system 140 may be a location-independent server system (e.g., cloud-based), which may be accessible by a variety of local computing devices 112 in geographically dispersed locations. Front-end server 142 may be in communication with back-end server 144 via wired or wireless communications link 143 over a local area network. Furthermore, although front-end server 142 and back-end server 144 are depicted in FIG. 1 as two discrete devices, those of ordinary skill in the art will recognize that the functionalities and services of those devices may be implemented on a single centralized device or, in the alternative, may be distributed across multiple discrete devices in geographically dispersed locations. Likewise, those of skill in the art will recognize that the representations of servers in the embodiments disclosed herein, and as shown in FIG. 1, are intended to cover both physical servers and virtual servers (or “virtual machines”).

In some embodiments, local computing device 112 can also communicate through network 130 with an on-premise computer system 152 located in a “controlled” environment 150, which can be a separate location from environment 120 and remote server system 140. According to some embodiments, for example, environment 150 can be a training or a physical therapy facility, in which athletic movement data of a user is acquired using a force plate 162 that is communicatively coupled with on-premise computer system 152. On-premise computer system 152 can extract selected portions of the acquired data and transmit it to remote server system 140, which, in turn, can normalize the selected portions of acquired data and determine an intervention. According to one aspect of the embodiments disclosed herein, local computing device 112 of environment 120 can transmit stored data directly to on-premise computer system 152, for example, in order to assess and compare athletic movement data between different environments for the same user. In other embodiments, local computing device 112 and on-premise computer system 152 can transmit data for user 105 to remote server system 140, which, in turn, can aggregate athletic movement data for user 105. Additional embodiments of on-premise computer systems, including devices and methods relating thereto, are further described in U.S. Pat. Nos. 9,223,855, 9,682,280, 9,737,758, U.S. patent application Ser. No. 14/050,735, and U.S. Patent Application Ser. No. 62/528,866, all of which are incorporated by reference herein in their entireties for all purposes.

Referring still to FIG. 1, in some embodiments, local computing device 112 may also synchronize a local database with the database 148 of a remote server system 140. In certain instances, this topology may be preferable, such as where heightened security is needed for local computing device 112 or the local area network on which local computing device 112 resides. For example, the owner of local computing device 112 may not want to permit any or some of the athletic movement data collected through local computing device 112 to be transmitted to the remote server system 140, which may be shared by multiple tenants. Under those circumstances, local computing device 112 may serve as a gateway, and conduct one-way synchronization or selective synchronization of the local database with database 148 of remote server system 140.

### Example Embodiment of Wearable Computing Device

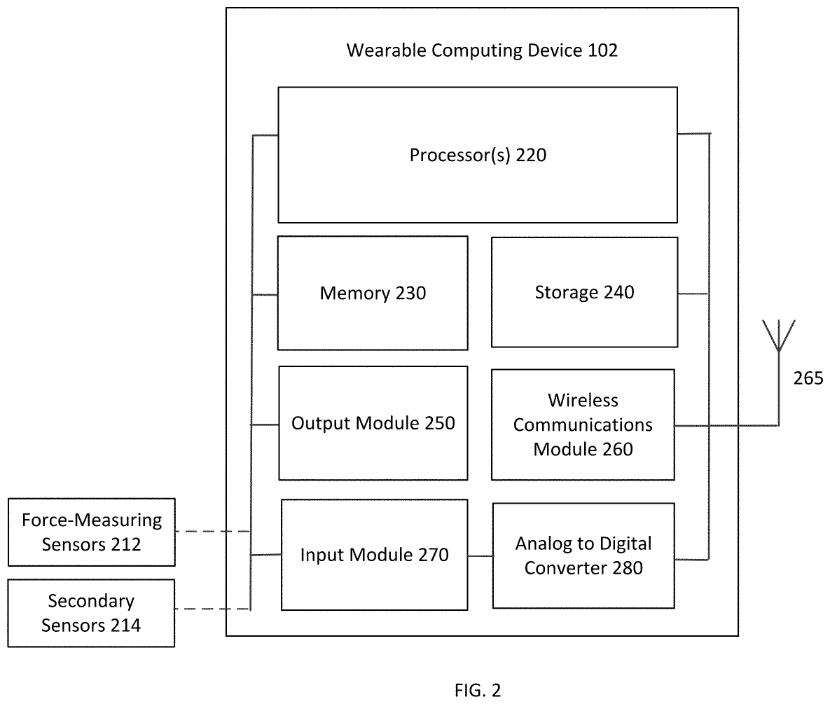

FIG. 2 is a block diagram depicting an example embodiment of a wearable computing device 102. Wearable computing device 102 may include one or more processors 220, which may comprise, for example, one or more of a general-purpose central processing unit (“CPU”), a graphics processing unit (“GPU”), an application-specific integrated circuit (“ASIC”), a field programmable gate array (“FPGA”), an Application-specific Standard Products (“ASSPs”), Systems-on-a-Chip (“SOCs”), Programmable Logic Devices (“PLDs”), or other similar components. Furthermore, processors 220 may include one or more processors, microprocessors, controllers, and/or microcontrollers, or a combination thereof, wherein each component may be a discrete chip or distributed amongst (and a portion of) a number of different chips, and collectively, may have the majority of the processing capability for acquiring, validating and analyzing athletic movement data. In many embodiments, wearable computing device 102 may also include one or more of the following components, each of which can be coupled to the one or more processors 220—memory 230, which may comprise non-transitory memory, RAM, Flash or other types of memory; mass storage devices 240; an output module 250; a wireless communications module 260 and an antenna 265 coupled thereto; an analog to digital converter module 280 configured to convert an analog signal received from the one or more sensors into a digital signal; and an input device module 270, which can comprise a port through which a user can couple a keyboard, keypad or memory device to upload, configure or upgrade software or firmware on the wearable computing device 102. In addition, in some embodiments, wearable computing device 102 can include a removable memory device, such as a Universal Flash Storage device, a micro SD memory card, an SD memory card, an SDHC memory card, an SDXC memory card, a CompactFlash memory card, or a memory stick. As understood by one of skill in the art, these components are electrically and communicatively coupled in a manner to make a functional device.

According to another aspect of the disclosed embodiments, wearable computing device 102 can also include one or more sensors coupled to the input device module 270, wherein the one or more sensors are configured to sense various characteristics of an athletic movement. For example, wearable computing device 102 can include one or more force-measuring sensors 212, configured to generate one or more signals in response to the detection of ground-reaction forces, such as those created during an athletic movement. In many of the embodiments, the force-measuring sensors 212 can comprise a piezoelectric material, such as lead zirconate titanate, barium titanate, sodium potassium niobate, potassium niobate, or sodium tungstate. Those of skill in the art will recognize that other materials having piezoelectric properties can be utilized, and that said materials are fully within the scope of the present disclosure. In other embodiments, the force-measuring sensors 212 can comprise on or more piezoresistive sensors, force-sensing resistors, thin-film strain gauge sensors, thin-film capacitive sensors, or any other type of sensor configured to measure force generated by an athletic movement.

According to another aspect of the embodiments disclosed herein, wearable computing device 102 can also include one or more secondary sensors 214 coupled to input module 270. For example, wearable computing device 102 can include one or more accelerometers for measuring acceleration, including but not limited to single- or three-axis accelerometers; magnetometers for measuring the Earth's magnetic field and a local magnetic field in order to determine the location and vector of a magnetic force; global positioning system (GPS) sensors; gyroscope sensors for measuring rotation and rotational velocity; or any other type of sensor configured to measure the velocity, acceleration, orientation, and/or position of wearable computing device 102. In other embodiments, secondary sensors 214 can also include temperature and pressure sensors for measuring environmental conditions. In many of the embodiments, the secondary sensors 214 can comprise microelectromechanical (MEMS) devices.

In some embodiments, instructions stored in memory 230 of wearable computing device 102, when executed by the processors 220, can cause processors 220 to corroborate signals received from force-measuring sensors 212 and secondary sensors 214. For example, signals received from force-measuring sensors 212 and the secondary sensors 214 may be time-synchronized and/or multiplexed by processors 220 of wearable computing device 102. In other embodiments, as described in further detail below, corroboration of sensor data can occur in local computing device 112 in addition to (or instead of) in wearable computing device 102.

Referring still to FIG. 2, in many embodiments, wearable computing device 102 can be configured to transmit data to a local computing device 112 via a wireless communications module 260. Wireless communications module 260 can be configured, for example, to wirelessly transmit stored sensor data (e.g., ground-reaction force values) to local computing device 112 according to a standard wireless networking protocol, such as 802.11x, Bluetooth, Bluetooth Low Energy, Near Field Communication (NFC), UHF, or infrared. In some embodiments, for example, wireless communications module 260 can be configured to transmit stored sensor data to local computing device 112, according to a NFC or Bluetooth protocol, when player 105 enters a predefined transmission range to local computing device 112. The proximity-based transmission can be initiated either by the wearable computing device 102 or local computing device 112. According to another aspect of the disclosed embodiments, stored sensor data can be transferred to local computing device 112 through a wired connection, such as through a micro USB port of the wearable computing device, or manually transferred using a removable memory device, such as a Universal Flash Storage device, a micro SD memory card, an SD memory card, an SDHC memory card, an SDXC memory card, a CompactFlash memory card, or a memory stick.

### Example Embodiment of Local Computing Device

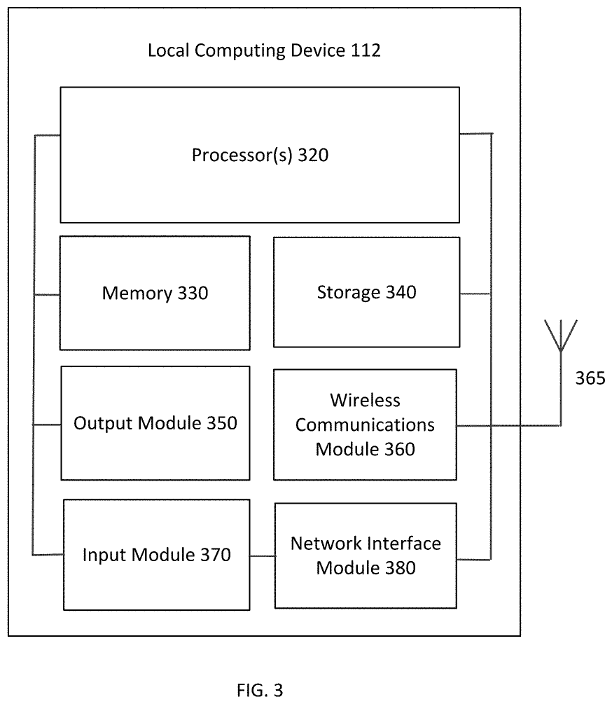

FIG. 3 is a block diagram depicting an example embodiment of local computing device 112. In many embodiments, local computing device 112 can be a personal computer, desktop computer, laptop computer or workstation. In other embodiments, local computing device 112 may also comprise a laptop computer, tablet computing device, smartphone, personal digital assistant, and/or other mobile computing devices. Referring to the block diagram of FIG. 3, local computing device 112 may include one or more processors 320, which may comprise, for example, one or more of a general-purpose CPU, a GPU, an ASIC, a FPGA, ASSPs, SOCs, PLDs, and other similar components. Processors 320 may comprise one or more processors, microprocessors, controllers, and/or microcontrollers, or a combination thereof, wherein each component may be a discrete chip or distributed amongst (and a portion of) a number of different chips, and collectively, may have the majority of the processing capability for receiving, processing, and transmitting athletic movement data. Local computing device 112 may also include memory 330, which may comprise non-transitory memory, RAM, Flash or other types of memory. Furthermore, local computing device 112 may include one or more mass storage devices 340, an output/display module 250, a wireless communications module 360 and an antenna 365 coupled thereto, one or more network interface modules 380, and an input module 370, which may include keyboards, mice, trackpads, touchpads, microphones and other user input devices, each of which may be communicatively coupled to local computing device 112 via a wired or wireless connection. As understood by one of skill in the art, these components are electrically and communicatively coupled in a manner to make a functional device.

In many of the embodiments of the present disclosure, wireless communications module 360 of local computing device 112 can be configured to communicate with one or more wearable computer devices, as previously described. In addition, a network interface module 380 can be configured to communicate with a remote server system 140. In some embodiments, wireless communications module 360 of local computing device 112 can be configured to communicate both with wearable computing device 102 and remote server system 140.

According to one aspect of the embodiments disclosed herein, local computing device 112 is configured to receive stored sensor data from one or more wearable computing devices. Furthermore, instructions stored in memory 330 of local computing device 112, when executed by processors 320, can cause processors 320 to extract a plurality of selected portions of sensor data. In some embodiments, for example, the stored sensor data can comprise stored ground-reaction force values, and the plurality of selected portions can comprise an average eccentric rate of force development, an average relative concentric force and a concentric relative impulse.

According to another aspect of the embodiments disclosed herein, the selected portions of sensor data can be transmitted by local computing device 112 to remote server system 140, which, in turn, returns one or more normalized values correlating to the selected portions of sensor data. The normalized values may be visually displayed through a user interface on local computing device 112. For example, in some embodiments, the normalized values may be depicted as T-scores in a vertical bar chart. In other embodiments, the one or more normalized values may be depicted as a plotted line as a function of time. These graphical user interfaces, as well as other visual representations, may be generated by processors 320 in response to instructions, e.g., in the form of a locally installed application, which resides in memory 330 of local computing device 112. Additional examples and descriptions of the processing of sensor data are described in U.S. Pat. Nos. 9,223,855, 9,682,280, 9,737,758, U.S. patent application Ser. No. 14/050,735, and U.S. Patent Application Ser. No. 62/528,866, all of which are incorporated by reference herein in their entireties for all purposes.

### Example Embodiments of Remote Server System

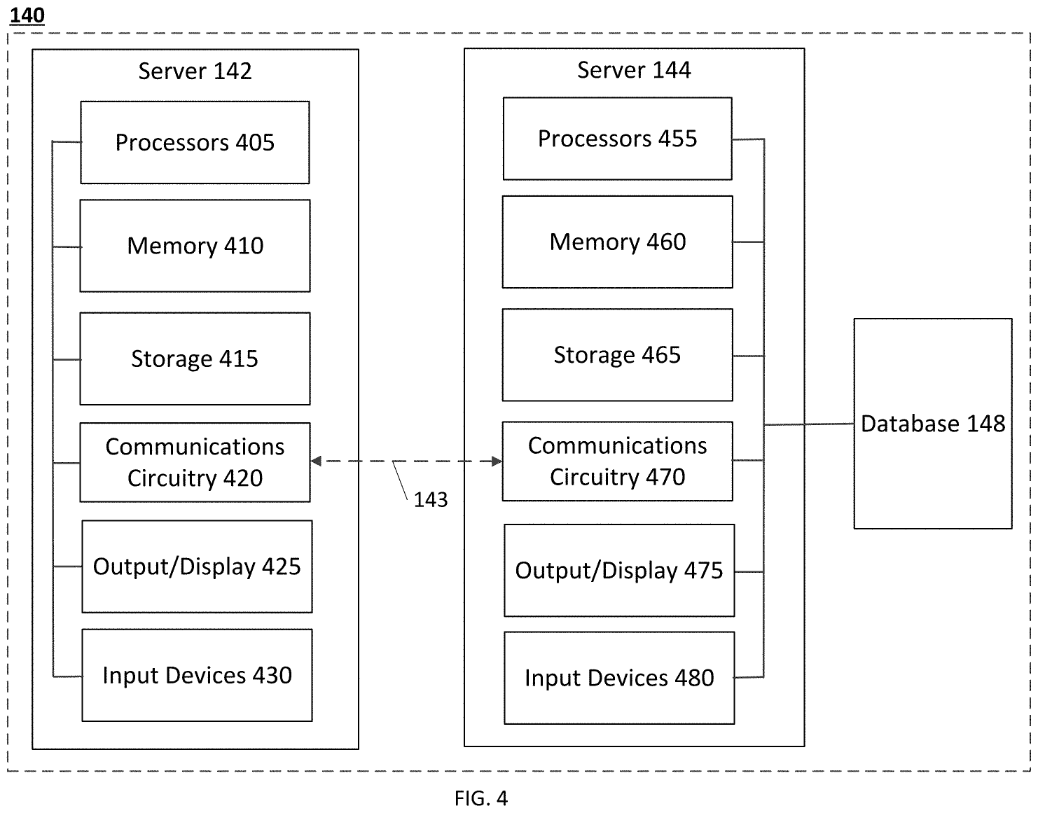

FIG. 4 is a block diagram depicting an example embodiment of remote server system 140 comprising one or more servers, and which may include a front-end server 142 and a back-end server 144. As shown in the diagram, servers 142, 144 may each include, respectively, an output/display module (425, 475), one or more processors (405, 455), memory (410, 460), including non-transitory memory, RAM, Flash or other types of memory, communications circuitry (420, 470), which may include both wireless and wired network interfaces, mass storage devices (415, 465), and input devices (430, 480), which may include keyboards, mice, trackpads, touchpads, microphones, and other user input devices. The one or more processors (405, 455) may include, for example, a general-purpose CPU, a GPU, an ASIC, an FPGA, ASSPs, SOCs, PLDs, and other similar components, and furthermore, may comprise one or more processors, microprocessors, controllers, and/or microcontrollers, each of which may be a discrete chip or distributed amongst (and a portion of) a number of different chips. As understood by one of skill in the art, these components can be electrically and communicatively coupled in a manner to make a functional device.

In some embodiments, front-end server 142 may be configured such that communications circuitry 420 provides for a single-network interface which allows front-end server 142 to communicate with the one or more local computing devices, as well as back-end server 144. In other embodiments, front-end server 142 may be configured such that communications circuitry 420 provides for two discrete network interfaces to provide for enhanced security, monitoring and traffic shaping and management. In addition, in most embodiments, front-end server 142 includes instructions stored in memory 410 that, when executed by the one or more processors 405, cause the one or more processors 405 to receive extracted portions of sensor data from one or more local computing devices, store the portions of sensor data to a database 148, and generate and transmit one or more normalized values associated with an athletic movement to a local computing device. In addition, the instructions stored in memory may further cause the one or more processors to perform one or more of the following routines: aggregate processed sensor data by various categories including by gender, by age, by body weight, by preferred sport and/or by position within a preferred sport; generate and store normalized values associated with an athletic movement for one or more of the aforementioned categories; update normalized values based on newly received processed sensor data from the one or more local computing devices; and perform synchronization between database 148 and one or more databases residing on one or more local server systems.

Referring still to FIG. 4, server 144 may include database 148 for storing selected portions of sensor data indicative of one or more characteristics of an athletic movement. In some embodiments, database 148 may reside on back-end server 144. In other embodiments, database 148 may be part of a storage area network, for example, to which back-end server 144 is communicatively coupled. Back-end server 144 may also include communications circuitry 470 which may be configured to facilitate communications to and from front-end server 142. Similar to the configuration of front-end server 142, in many embodiments, communications circuitry 470 may include a single network interface, either wired or wireless; or, in other embodiments, communications circuitry 470 may include multiple network interfaces, either wired or wireless, to provide for enhanced security, monitoring and traffic shaping and management.

### Example Embodiments of Wearable Computing Devices and Sensor Configurations

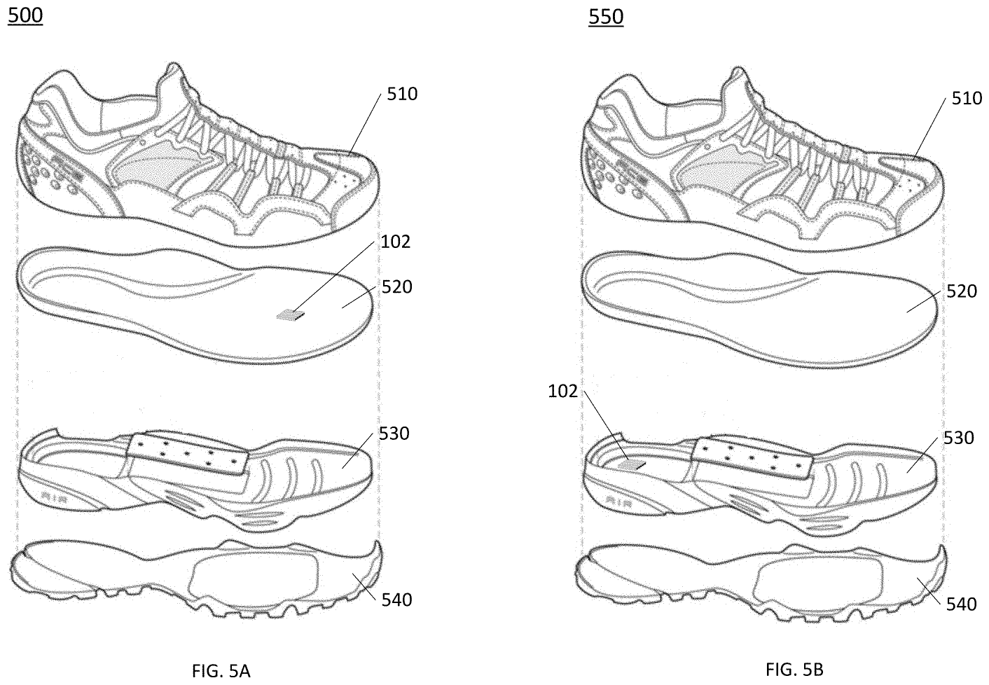

FIGS. 5A and 5B illustrate two exemplary embodiments of configurations for a wearable computing device 102. FIGS. 5A and 5B each depict an exploded and perspective view of a shoe comprising an upper portion 510 for protecting and holding in place the top portion of a user's foot; an insole 520 for providing arch support; a midsole 530 for absorbing shock and providing additional support; and an outsole 540 for providing traction, protection and additional shock absorption. Upper portion 510 can include laces, a tongue and a collar, and may be constructed at least in part from mesh materials to increase ventilation around the foot. In many embodiments, upper portion 510 can also be constructed at least in part of leather, suede or a similar durable fabric. Insole 520 can be a removable insert that is disposed directly beneath the foot (when worn) and also disposed on top of midsole 530. Insole 520 can provide for comfort, cushioning and support of the user's foot. Midsole 530 is disposed at least partially under insole 520, and can include a plurality of air pockets or gel materials for shock absorption purposes. Midsole 530 can be manufactured from ethyl vinyl acetate (EVA), polyurethane foam, or other materials having similar properties to hard foam. Outsole 540 is disposed on the bottom of the shoe, and can be manufactured from a rubber material. In addition, outsole 540 can include a plurality of grooves and/or textured surfaces to provide for additional traction.

FIG. 5A depicts a shoe in which wearable computing device 102 is disposed in insole 520. In some embodiments, locating wearable computing device 102 in insole 520 is advantageous in that insole 520 can typically be removed and/or replaced with ease. Additionally, although not shown here, one or more sensors may be easily re-configured on insole 520 to accommodate for varying foot sizes and shapes. According to one aspect of the disclosed embodiments, for example, wearable computing device 102 can be manufactured and sold as a kit with insole to be used in any shoe of the user's choosing.

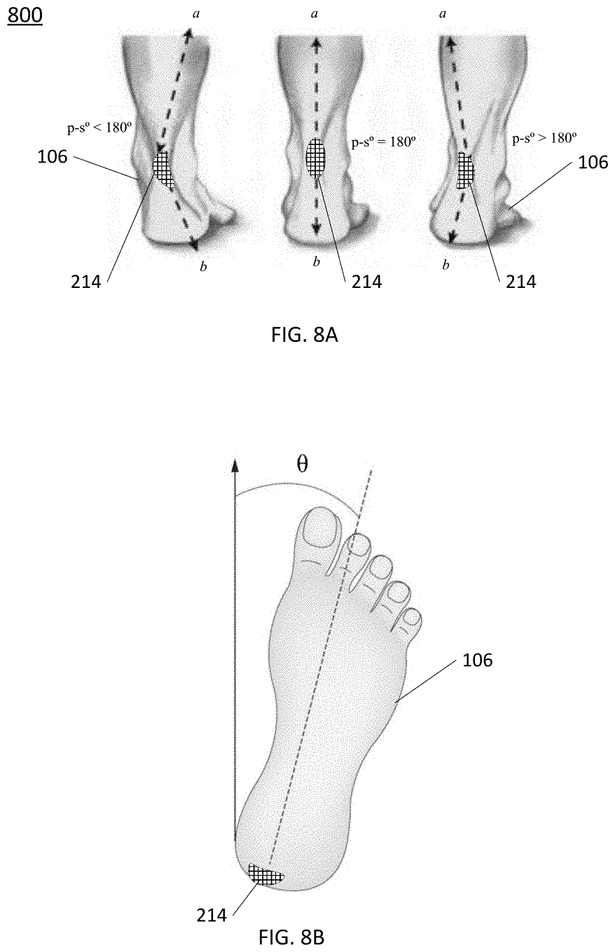

FIG. 5B depicts a shoe in which wearable computing device 102 is disposed in midsole 530. Locating wearable computing device 102 in midsole 530 may be preferable in some embodiments where durability is desired, as midsole 530 can be less susceptible to wear-and-tear, as well as environmental factors (e.g., dampness), as compared to insole 520. Additionally, in some embodiments, locating wearable computing device 102 in midsole 530 may be preferable to detect pronation and/or supination of the foot, as described below with respect to FIG. 8A.

Although wearable computing device 102 is depicted in a single location in FIGS. 5A and 5B, those of skill in the art will appreciate that various components of wearable computing device 102 can also be disposed in different locations within a single portion of a shoe, or disposed within different locations within different portions of the shoe. For example, according to some embodiments, processors and memory of wearable computing device 102 can be disposed in midsole 530 or outsole 540, and force-measuring sensors can be disposed in insole 520. In other embodiments, secondary sensors can be separately disposed in upper portion 510 or midsole 530. As another example, according to some embodiments, insole 520 can include force-measuring sensors (and, optionally, secondary sensors), which include one or more electrical contacts, and where at least a portion of the electrical contacts are exposed on the underside of insole 520. Midsole 530 can include processors, memory, wireless communication module and any of the other components of wearable computing device 102 described with respect to FIG. 2, and further include a corresponding set of electrical contacts that are adapted to be coupled with the electrical contacts of insole 520, when insole 520 is properly inserted into the shoe. Other combinations and permutations are possible, and those of skill in the art will recognize that these are fully within the scope of the present disclosure.

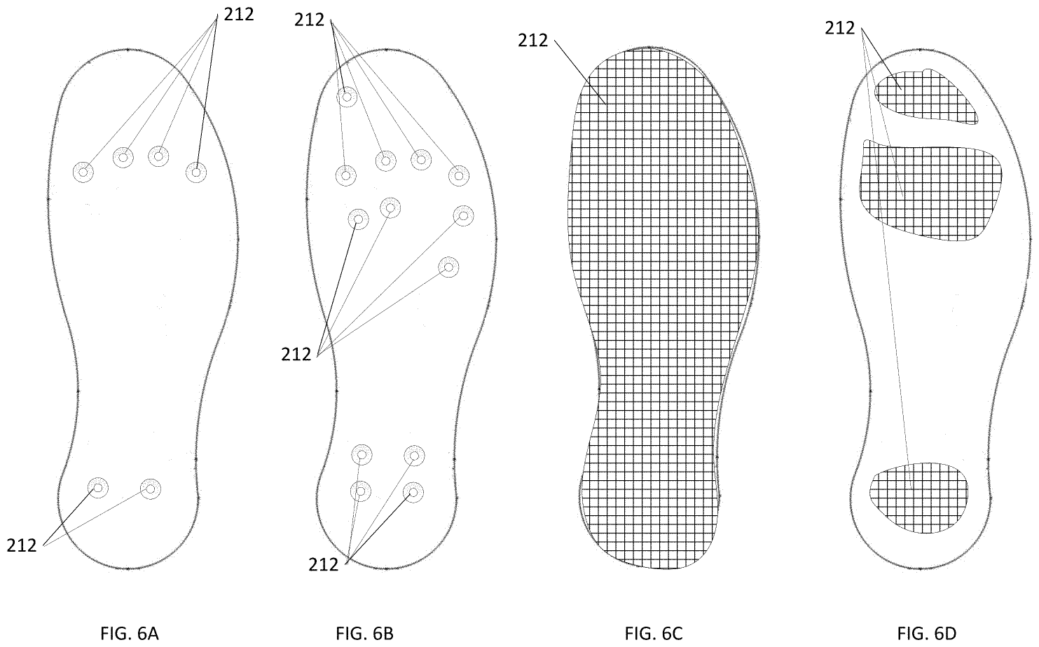

FIGS. 6A to 6D are sectional top views of example embodiments of wearable computing devices with various sensor configurations. According to one aspect of the embodiments disclosed herein, and as described above, one or more sensors 212 can be disposed in one or more portions of a shoe, such as an upper portion, an insole, a midsole, or an outsole. The sectional top views depicted in FIGS. 6A to 6D are not meant to be limiting to one particular portion of the shoe, and are meant to show exemplary cross-sectional configurations of the one or more sensors of wearable computing device. The sensors 212 depicted in FIGS. 6A to 6D can comprise one or more different types of force-measuring sensors, including but not limited to, piezoelectric force sensors, piezoresistive force sensors, force-sensing resistors, thin-film strain gauge sensors, thin-film capacitive sensors, or any other type of sensors adapted to sense ground-reaction forces. Furthermore, in many of the embodiments, sensors 212 can be configured to measure ground-reaction forces at discrete points, as depicted in FIGS. 6A and 6B, which can comprise, for example, piezoelectric force sensors. In other embodiments, sensors 212 can comprise a “grid,” as depicted in FIGS. 6C and 6D, which can comprise, for example, an array of capacitive sensors. With respect to FIGS. 6A and 6B, six-sensor and thirteen-sensor configurations are depicted. Those of skill in the art will appreciate that other numbers and placements of sensors can be utilized and are fully within the scope of the present disclosure. Similarly, FIG. 6C depicts a sensor grid covering the entirety of the cross-sectional area of the foot, and FIG. 6D depicts a three-sensor grid configuration covering portions of the cross-sectional area of the foot. Those of skill in the art will appreciate that other numbers and geometries for sensor grids can be utilized and are fully within the scope of the present disclosure.

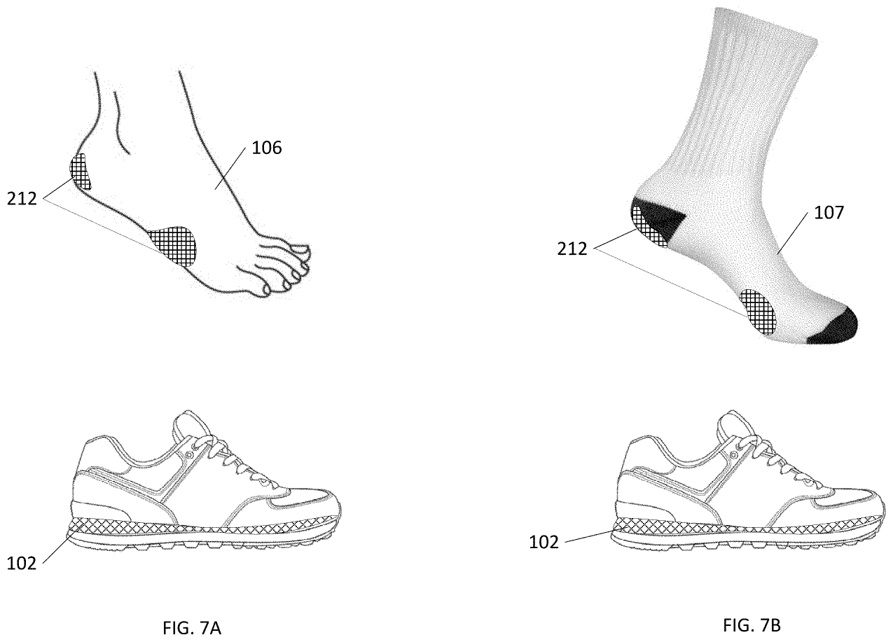

FIGS. 7A and 7B illustrate two alternative exemplary configurations of wearable computing devices 102. FIG. 7A depicts an exploded and perspective view of a wearable computing device, of which at least a portion of is disposed in one or more adhesive patches configured to be applied to an outer skin surface of the foot 106. In some embodiments, for example, one or more adhesive patches can include sensors 212, which can be force-measuring sensors to sense ground-reaction forces at discrete points (as shown in FIGS. 6A and 6B), or force-measuring sensor grids (as shown in FIGS. 6C and 6D). According to one aspect of the disclosed embodiments, the adhesive patches can also include one or more electrical contacts (not shown) configured to be removably coupled to a corresponding set of electrical contacts (not shown) disposed on the insole of the shoe. The corresponding set of electrical contacts on the insole of the shoe can be coupled to the processors and memory of the wearable computing device, which can be disposed in the insole, midsole, or outsole of the shoe. In this manner, various electrical components can be housed in a portion of the shoe, such as in the insole, midsole or outsole, while sensors 212 can be housed in the one or more adhesive patches to be applied to the foot 106. Such a configuration may be advantageous in the sense that the one or more adhesive patches can allow for customizable sensor placement.

FIG. 7B depicts an exploded and perspective view of another wearable computing device, of which at least a portion of is disposed in a sock 107. In some embodiments, for example, one or more sensors 212 can be embedded, attached to or woven within the fabric of an athletic sock 107, as shown in FIG. 7B. As with other embodiments, sensors 212 can be force-measuring sensors to sense ground-reaction forces at discrete points (as shown in FIGS. 6A and 6B), or force-measuring sensor grids (as shown in FIGS. 6C and 6D). Similar to the embodiment described with respect to FIG. 7A, sensors 212 of FIG. 7B can include one or more electrical contacts (not shown) configured to be removably coupled to a corresponding set of electrical contacts (not shown) disposed on the insole of the shoe. The corresponding set of electrical contacts on the insole of the shoe can be coupled to the processors and memory of the wearable computing device, which can be disposed in the insole, midsole, or outsole of the shoe. In addition, except for a portion of the electrical contacts, sensors 212 can be enclosed within a durable waterproof or water-resistant covering. In this manner, sock 107 with sensors 212 can be reused without having to replace the sensors each time.

FIG. 8A is a back view of another embodiment of a wearable computing device, as shown in three different stages. In some embodiments, wearable computing device can be configured to detect and measure a degree of pronation or supination of the foot 106. For example, as shown in FIG. 8A, one or more secondary sensors 214 can be attached to a heel portion of foot 106, such as along the Achilles tendon. The secondary sensors 214 can include, for example, one or more gyroscopic sensors, magnetometers, accelerometers, piezoresistive sensors, force-sensing resistors, or thin-film capacitive sensors. As can be seen at the left portion of FIG. 8A, foot 106 is shown in a pronated position, wherein angle, p-s°, is created between axis a and axis b, and wherein angle, p-s°, is less than 180 degrees. As shown in the center portion of FIG. 8A, foot 106 is shown in a neutral position, wherein angle, p-s°, is equal or substantially equal to 180 degrees. As shown in the right portion of FIG. 8A, foot 106 is shown in a supinated position, wherein angle, p-s°, is greater than 180 degrees. Thus, according to one aspect of the embodiments disclosed herein, sensor 214 can determine whether the foot is in a pronated, neutral or supinated position, and also determine the degree to which the foot is in either a pronated or supinated position.

In some embodiments, for example, instructions stored in memory of wearable computing device, when executed by the processors, can cause the processors to determine one or more pronation-supination values associated with the pronation or supination of the foot (e.g., p-s°), and in response to one or more pronation-supination values exceeding one or more predetermined pronation-supination thresholds, storing the one or more pronation-supination values in memory of the wearable computing device. In some embodiments, the pronation-supination values can be determined using one or more signals generated by secondary sensors 214. In other embodiments, the pronation-supination values can be determined using one or more signals generated by force-measuring sensors 212 (not shown), for example, by determining the displacement and/or velocity of a center of pressure. In still other embodiments, the pronation-supination values can be determined using a combination of signals received from the force-measuring sensors 212 (not shown) and secondary sensors 214, wherein signals from different sensors and sensor types can be correlated by a time stamp associated with each signal that is provided by a GPS sensor.

FIG. 8B is a top view depicting another example embodiment of a wearable computing device. In some embodiments, wearable computing device can be configured to detect and measure a degree of rotation of the foot 106. For example, as shown in FIG. 8B, one or more secondary sensors 214 can be attached to a heel portion of foot 106. The secondary sensors 214 can include, for example, one or more gyroscopic sensors, magnetometers, accelerometers, piezoresistive sensors, force-sensing resistors, or thin-film capacitive sensors. As can be seen in FIG. 8B, sensor 214 can determine whether foot 106 is rotated from a neutral position, and also determine the degree, θ, to which the foot is rotated. For example, in some embodiments, instructions stored in memory of wearable computing device, when executed by the processors, can cause the processors to determine one or more rotational values (e.g., 0) associated with the rotation of the foot 106, and in response to one or more rotational values exceeding one or more predetermined foot rotation thresholds, storing the one or more rotational values in memory of the wearable computing device. In some embodiments, the rotational values can be determined using one or more signals generated by secondary sensors 214. In other embodiments, the rotational values can be determined using one or more signals generated by force-measuring sensors 212 (not shown), for example, by determining the displacement and/or velocity of a center of pressure. In still other embodiments, the rotational values can be determined using a combination of signals received from the force-measuring sensors 212 (not shown) and secondary sensors 214, wherein signals from different sensors and sensor types can be correlated by a time stamp associated with each signal that is provided by a GPS sensor.

### Example Embodiments of Methods for Acquiring and Analyzing Athletic Movement Data

Example embodiments of methods for acquiring athletic movement data will now be described. Those of skill in the art will understand that the method steps disclosed herein may comprise instructions stored in memory of a wearable computing device and/or local computing device, and that the instructions, when executed by the one or more processors of the wearable or local computing device, may cause the one or more processors to perform the steps disclosed herein. In addition, many of the method steps and functions are described herein as being performed by a wearable computing device, local computer system, or remote server system. It will be clearly understood by those of skill in the art that these descriptions are meant to be merely illustrative and non-limiting, and that the method steps and functions can be performed by any of the wearable computing device, local computer system, and remote server system, or any combination thereof.

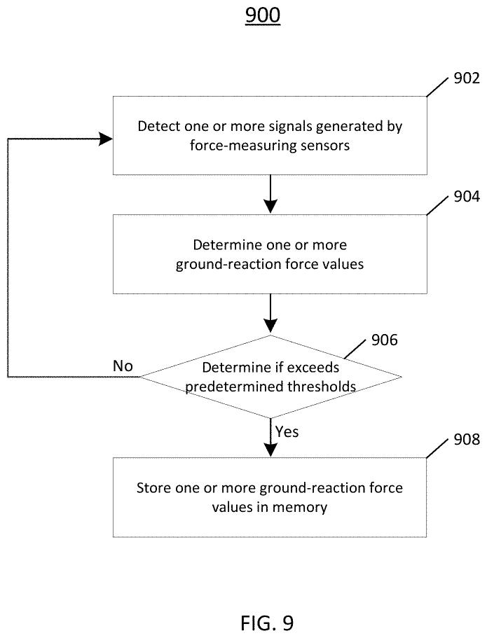

Referring to FIG. 9, a flow diagram is provided, depicting an overview of an example embodiment of a method 900 for acquiring athletic movement data. Before proceeding to Step 902 of FIG. 9, one or more additional of the following steps can be implemented. First, the wearable computing device can be configured to default to a low-power or “sleep” state when not in use. In the “sleep” state, one or more of the electronic components of the wearable computing device, including the wireless communications module, output module and others, can be powered off or set to a low-power mode. Second, the wearable computer device can be configured to detect the presence of a foot, e.g., when a user wears the shoe, and enters into a “idle” state. The “idle” state can be initiated, for example, by a minimum weight being detected by one or more force-measuring sensors. In other embodiments, the “idle” state can be initiated by the closure of a circuit by coupling the electrical contacts of the sensors with the electrical contacts of the insole, as described earlier with respect to FIG. 7A. In still other embodiments, the “idle” state can be initiated by a pressure switch on the insole, a manual switch or button disposed on the outsole, or any mechanism by which the shoe can be powered on. In the “idle” state, most electronic components of the wearable computing device, including the processors, force-measuring sensors, secondary sensors, wireless communications module, input modules, and output modules, can be powered on. In the “idle” state, however, signals generated by sensors may not be stored in memory. Additionally, the wearable computing device can perform a “weight validation” check upon entering the “idle” state. Using the one or more force-measuring sensors, wearable computing device can determine whether the weight of the user is within a threshold percentage of the intended user. If the measured weight is not within the threshold percentage of the intended user, the wearable computing device can revert back to the “sleep” state. In this manner, the identity of the user can be verified. Finally, wearable computing device can be configured to enter into an “active” state if the “weight validation” check is passed, and, optionally, if a minimum velocity or force is detected. In the “active” state, the wearable computing device is prepared to store sensor data that meets one or more predetermined thresholds, as will be described below.

Turning to FIG. 9, at Step 902, one or more signals generated by force-measuring sensors are detected by a wearable computing device. From the signals, at Step 904, one or more ground-reaction force values are determined by the one or more processors and an analog-to-digital converter of the wearable computing device. The one or more ground-reaction force values are compared to one or more predetermined ground-reaction force thresholds at Step 906. If the ground-reaction force values exceed one or more predetermined ground-reaction force thresholds, the ground-reaction force values are stored in memory of the wearable computing device at Step 908.

According to one aspect of the embodiments disclosed herein, the predetermined ground-reaction force thresholds can include, for example, one or more of the following: a single ground-reaction force value above a certain threshold value; a predetermined number (e.g., 2, 3, 4, 5 or 6) of consecutive ground-reaction force values above a certain threshold value; a determined running average of ground-reaction force values above a certain threshold; or a determined median of ground-reaction force values above a certain threshold. In some embodiments, a predetermined ground-reaction force threshold may also include determining when ground-reaction force values in conjunction with secondary sensor values are above a plurality of thresholds. Those of skill in the art will recognize that other conditions for the storage of sensor data, or combinations of conditions, are possible, and fully within the scope of the present disclosure.

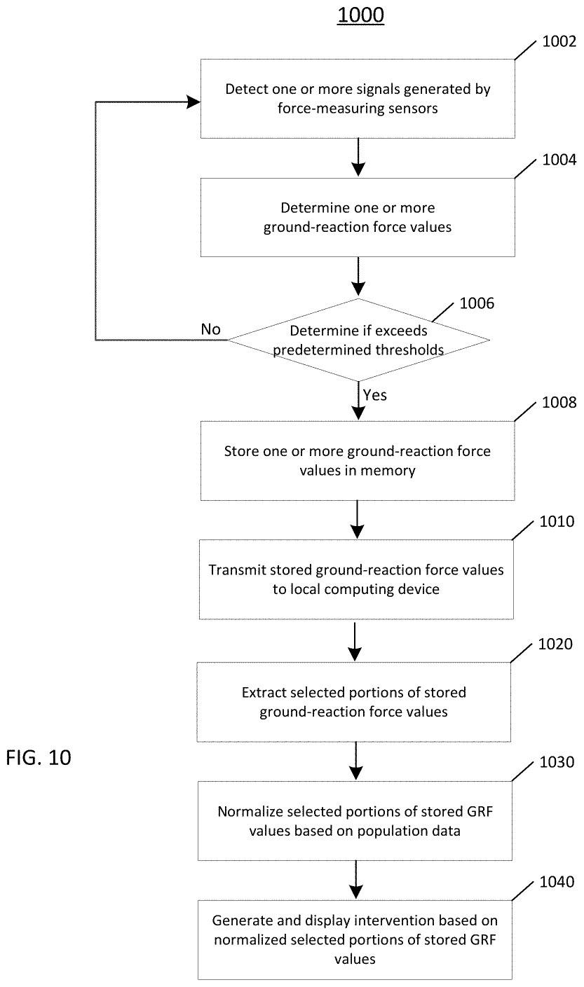

Turning to FIG. 10, a flow diagram is provided, depicting an overview of another example embodiment of a method 1000 for acquiring athletic movement data. As with the previous example embodiment of a method described with respect to FIG. 9, before proceeding to Step 1002, one or more of the following steps relating to a “sleep” state, an “idle” state, a “weight validation” check, or an “active” state can be performed. Additionally, as can be seen in FIG. 10, Steps 1002, 1004, 1006, and 1008 are similar to Steps 902, 904, 906, and 908 of the exemplary method described with respect to FIG. 9. Proceeding to Step 1010, after one or more ground-reaction force values are stored in memory of the wearable computing device, the stored ground-reaction force values can be transmitted to a local computing device. As described earlier, the transmission can be wireless, i.e., transmitted by the wireless communications module of the wearable computing device to the local computing device according to a standard wireless networking protocol (e.g., 802.11x, Bluetooth or Bluetooth Low Energy, NFC, UHF or infrared). Moreover, in some embodiments, the transmission can occur periodically according to a schedule, or can be initiated when the wearable computing device enters a predefined transmission range of the local computing device, such as according to a Near Field Communication protocol. In other embodiments, transmission can occur by a wired link, such as by a USB cable connecting to a physical port disposed in the shoe. In still other embodiments, transmission can occur manually by transferring a removable media device, such as a Universal Flash Storage device, a micro SD memory card, an SD memory card, an SDHC memory card, an SDXC memory card, a CompactFlash memory card, or a memory stick, from the wearable computing device to the local computing device.

Referring still to FIG. 10, at Step 1020, instructions stored in memory of the local computing device, when executed by the processors of the local computing device, can cause the processors to extract selected portions of ground-reaction force values. In some embodiments, for example, the plurality of selected portions can comprise an average eccentric rate of force development, an average relative concentric force and a concentric relative impulse. At Step 1030, the selected portions can be normalized based on population data in a database. In some embodiments, for example, selected portions of the sensor data are transmitted to a remote server system that can include, or be communicatively coupled with a database comprising population data. The remote server system can determine normalized values corresponding to the received selected portions based on population data in the database, and then transmit the normalized values to the local computing device. In some embodiments, the database may comprise data for an entire population of athletes, which can be used to determine normalized values of the selected portions of sensor data. In other embodiments, a subset of the entire population of athletes in the database is used to determine normalized values for the selected portions of sensor data. For example, normalized values may be based in part on average ground-reaction force values for athletes in the same sport as the user. Other subsets of athletes may include gender, body weight range, age range, injury type, and/or a position within a preferred sport. Those of skill in the art will appreciate that these examples are not meant to be exhaustive, and that other subsets of athletes within the database are fully within the scope of the present disclosure. In addition, in many of the embodiments of the present disclosure, the determination of the normalized values may be performed by the one or more processors of the remote server system by either of the front-end server or the back-end server. In other embodiments, the determination of normalized values may be performed elsewhere, such as, for example, the local computing device.

According to another aspect of many of the embodiments disclosed herein, the normalized values can comprise T-scores. T-scores enable a user to take a raw value (e.g., the sensor data) and transform it into a standardized score that allows the user to contextualize his or her assessment within a relevant population of athletes. A standardized score is typically determined by using the mean and standard deviation values from the relevant population data, as represented by the following equation:

$$z = \frac{x - μ}{σ}$$

where z is the standard score, x is the raw score (i.e., processed sensor data), p is the mean of the relevant population, and a is the standard deviation of the relevant population. The T-score is a standard z score shifted and scaled to have a mean of 50 and a standard deviation of 10. A standard z score may be converted to a T-score by the following equation:

T=(z×10)+50

In this regard, T-scores are both meaningful and easy to comprehend. Unlike other standardized measures (e.g., z-scores), T-scores are always positive and typically comprise whole integers. In addition, a T-score of over 50 is above average, a T-score of below 50 is below average, and each increment of 10 represents one standard deviation away from the mean value.

Referring back to FIG. 10, at Step 1040, an intervention can be generated and displayed based on the normalized portions of ground-reaction force values. In some embodiments, for example, interventions can include a recommended regimen of training stored in the database of remote server system and associated with one or more of the normalized portions of ground-reaction force values. Other interventions are discussed in U.S. patent application Ser. No. 14/050,735, which is incorporated by reference herein in its entirety for all purposes.

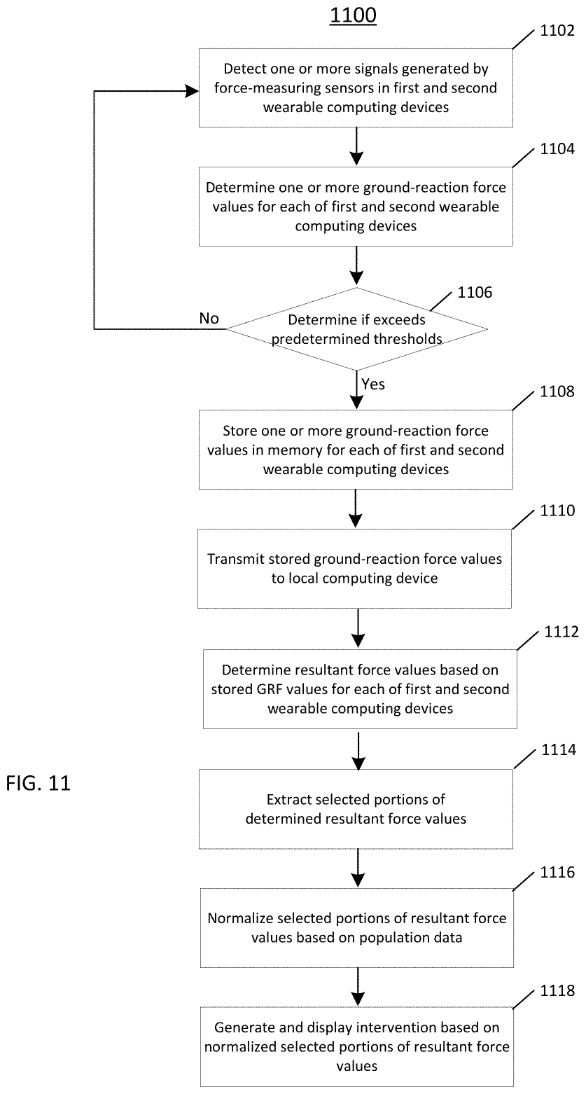

Turning to FIG. 11, a flow diagram is provided, depicting an overview of another example embodiment of a method 1000 for acquiring athletic movement data using multiple wearable computing devices (e.g., one wearable computing device on each foot of the user). As with the previous example embodiments of methods, before proceeding to Step 1102, one or more of the aforementioned steps relating to a “sleep” state, an “idle” state, a “weight validation” check, and an “active” state can be performed. Additionally, Steps 1102, 1104, 1106, 1108 and 1110 of FIG. 11 are similar to the steps described in the previous exemplary method of FIG. 10.

Turning to Step 1112, after the stored ground-reaction force values from each wearable computing device is transmitted to the local computing device, resultant force values can be determined. According to one aspect of the embodiments disclosed herein, a resultant force from the ground-reaction force values of each wearable computing device. For example, the following equation can be used to determine F_{R}, the resultant force, where Fₓ is the ground-reaction force from a first wearable computing device, F_{y} is the ground-reaction force from a second wearable computing device, and θ_{Fx,Fy} is the angle between forces, Fₓ and F_{y}:

F_{R}=√Fₓ²+F_{y}²−2(Fₓ)(F_{y})cos(180−θ_{Fx,Fy})

At Step 1114, instructions stored in memory of the local computing device, when executed by the processors of the local computing device, can cause the processors to extract selected portions of the resultant ground-reaction force values. In some embodiments, for example, the plurality of selected portions can comprise an average eccentric rate of force development, an average relative concentric force and a concentric relative impulse. Like the steps described in the exemplary embodiments methods of FIG. 10, at Step 1116 and 1118, the selected portions can be normalized based on population data in a database, and an intervention can be generated and displayed. In many of the embodiments, the normalized values can comprise T-scores.

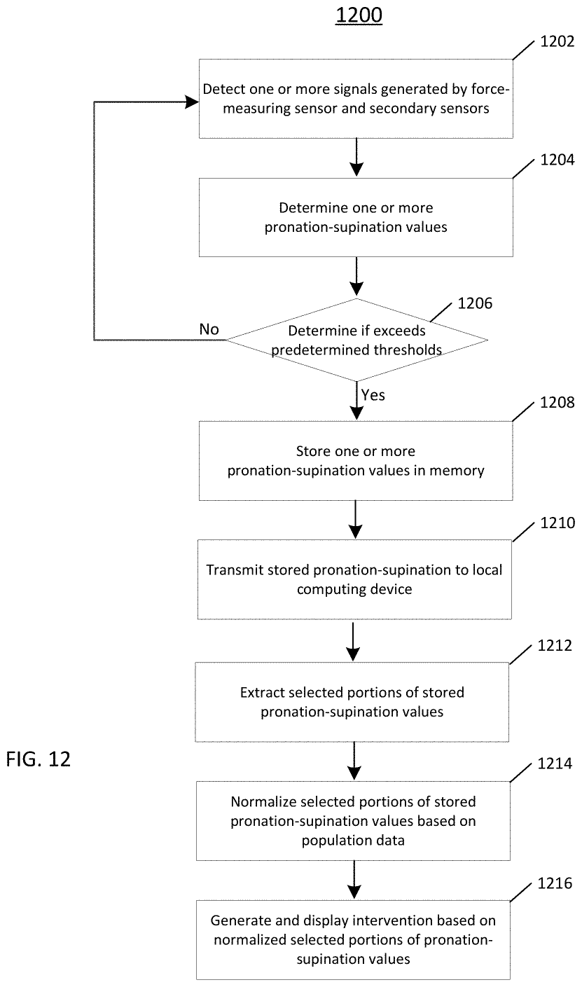

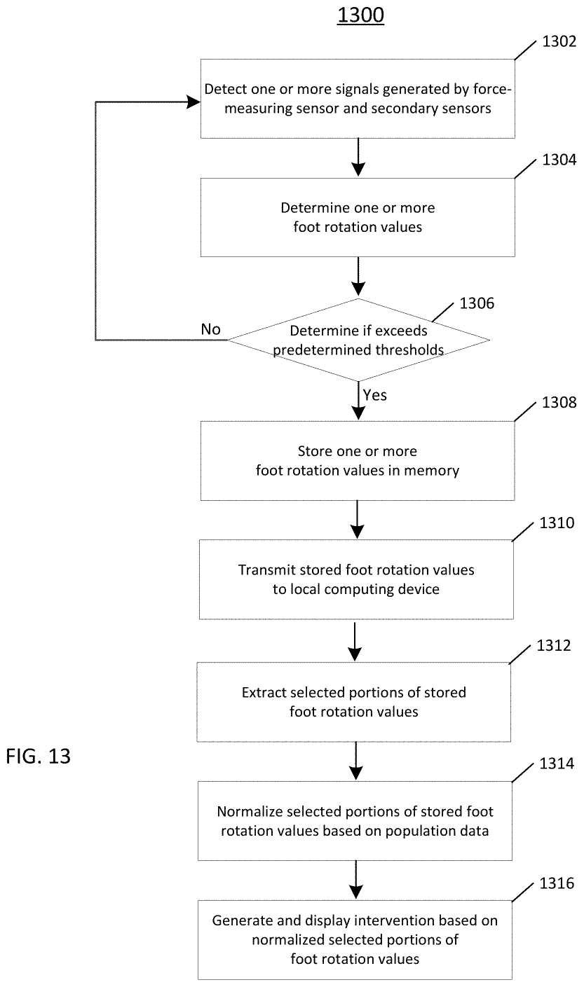

In FIGS. 12 and 13, flow chart diagrams are provided, depicting overviews of additional example embodiments of methods 1200 and 1300, respectively, for acquiring athletic movement data relating to pronation-supination and foot rotation of a user's foot. As with the previous embodiments, methods 1200 and 1300 can also include one or more of the aforementioned steps relating to a “sleep” state, an “idle” state, a “weight validation” check, and an “active” state. The method steps of exemplary methods 1200 and 1300 are analogous to that of method 1000, except that these methods can utilize signals generated by either or both of the force-measuring sensors and secondary sensors. For example, in some embodiments, at Step 1204 of method 1200, pronation-supination values can be determined using signals received from force-measuring sensors by determining displacement and velocity values for a center of pressure. In other embodiments, pronation-supination values can be determined at Step 1204 by using signals received from secondary sensors, such as gyroscope sensors, accelerometers, magnetometers and the like. In still other embodiments, pronation-supination values can be determined at Step 1204 by corroborating signals received from both force-measuring sensors and secondary sensors using time-stamps, for example, generated by a GPS sensor. Similarly, with respect to method 1300, foot rotation values can be determined using force-measuring sensors, secondary sensors (e.g., gyroscope sensors, accelerometers, or magnetometers), or a combination thereof. Those of skill in the art will appreciate that additional sensor data, such as that from temperature and/or pressure sensors, can be utilized to further corroborate the determined values.

### Additional Example Embodiments of Wearable Computing Devices

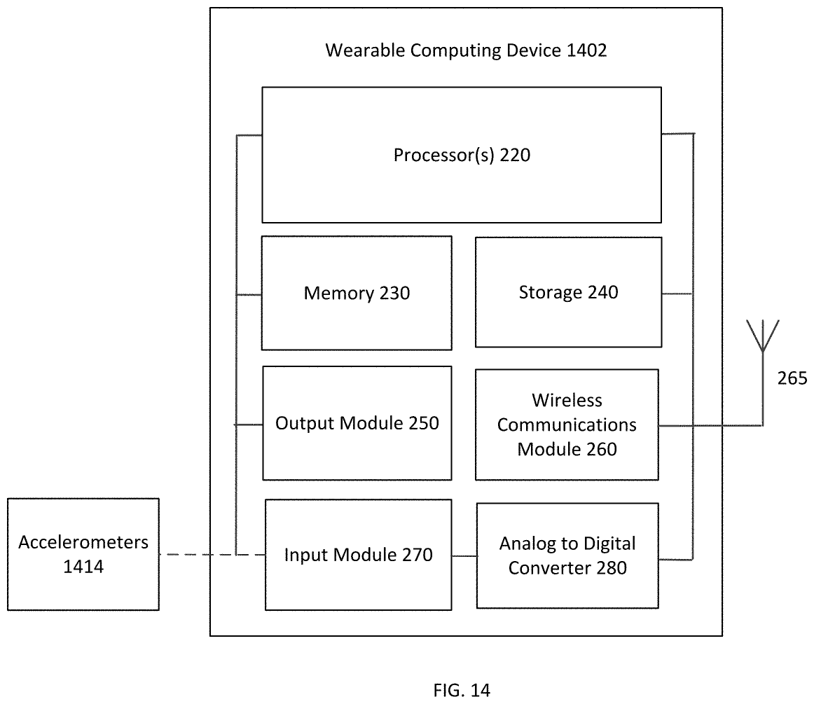

FIG. 14 is a block diagram depicting another embodiment of a wearable computing device 1402. Similar to the wearable computing device 102 described with respect to FIG. 2, wearable computing device 1402 can include one or more processors 220, which may comprise one or more of a CPU, a GPU, an ASIC, an FPGA, an ASSP, an SOC, a PLD, or other similar components. Processors 220 can include one or more processors, microprocessors, controllers, and/or microcontrollers, or a combination thereof, wherein each component may be a discrete chip or distributed among a number of different chips, and collectively, may have the majority of the processing capability for acquiring, validating and analyzing athletic movement data.

Wearable computing device 1402 may also include one or more of memory 230, which may comprise non-transitory memory, RAM, Flash or other types of memory; mass storage devices 240; an output module 250; a wireless communications module 260 and an antenna 265 coupled thereto; an analog to digital converter module 280 configured to convert an analog signal received from accelerometers 1414 into a digital signal; and an input device module 270, which can comprise a port through which a user can couple a keyboard, keypad or memory device to upload, configure or upgrade software or firmware on the wearable computing device 1402.

Wearable computing device 1402 can also include a removable memory device, such as a Universal Flash Storage device, a micro SD memory card, an SD memory card, an SDHC memory card, an SDXC memory card, a CompactFlash memory card, or a memory stick. As understood by one of skill in the art, these components are electrically and communicatively coupled in a manner to make a functional device.

According to one aspect of the embodiments, wearable computing device 1402 is distinct from wearable computing device 102 in that it includes one or more accelerometers 1414 coupled to the input device module 270, and does not include force-measuring sensors. Accelerometers 1414 can include single- or three-axis accelerometers, which can also comprise microelectromechanical (MEMS) devices. Accelerometers 1414 can be configured to sense various characteristics of athletic movements, which are described below.

According to another aspect of these embodiments, instructions stored in memory 230 of wearable computing device 1402, when executed by the processors 220, can cause processors 220 to process signals received from accelerometers 1414 to determine various characteristics of the user's athletic movements. These characteristics can include, for example, how fast an athlete is moving, how many strides are taken by each leg of the athlete, average step length, amount of inactivity, how well the athlete can maneuver turns, how balanced the athlete is with respect to weight distribution, how much time an athlete is spent in contact with the ground, and how well the athlete is accelerating. This list of characteristics is not meant to be exhaustive, and those of skill in the art will understand that other characteristics of athletic movements can be determined using the signal data received from accelerometers 1414. Those of skill in the art will also recognize that while the characteristics are described in terms of athletic movement, they can have significance in other fields, such as medicine, physical therapy, and military applications, to name only a few.

Referring still to FIG. 14, in many embodiments, wearable computing device 1402 can be configured to transmit data to a local computing device 112 via a wireless communications module 260. Wireless communications module 260 can be configured, for example, to wirelessly transmit stored characteristics of athletic movements to local computing device 112 according to a standard wireless networking protocol, such as 802.11x, Bluetooth, Bluetooth Low Energy, Near Field Communication (NFC), UHF, or infrared. According to another aspect of the disclosed embodiments, stored sensor data can be transferred to local computing device 112 through a wired connection, such as through a micro USB port of the wearable computing device, or manually transferred using a removable memory device, such as a Universal Flash Storage device, a micro SD memory card, an SD memory card, an SDHC memory card, an SDXC memory card, a CompactFlash memory card, or a memory stick.

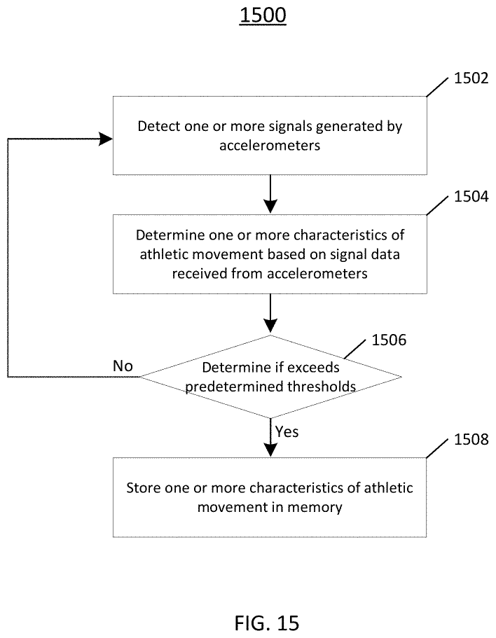

Turning to FIG. 15, a flow diagram is provided, depicting an overview of an example embodiment of a method 1500 for acquiring athletic movement data, which can be implemented using the wearable computing device 1402 of FIG. 14. Before proceeding to Step 1502 of FIG. 15, those of skill in the art will understand that the various states (“sleep,” “idle,” and “active”) and validation checks that were previously described with respect to FIG. 9, for example, can also be implemented with the following embodiment.

Referring now to FIG. 15, at Step 1502, one or more signals generated by accelerometers are detected by wearable computing device 1402. From the signals, at Step 1504, one or more characteristics of athletic movement can be determined by the one or more processors and an analog-to-digital converter of the wearable computing device 1402. In some embodiments, the one or more characteristics can include one or more of: how fast an athlete is moving, how many strides are taken by each leg of the athlete, average step length, amount of inactivity, how well the athlete can maneuver turns, how balanced the athlete is with respect to weight distribution, how much time an athlete is spent in contact with the ground, and how well the athlete is accelerating. As stated earlier, this list of characteristics is not meant to be exhaustive, and those of skill in the art will understand that other characteristics of athletic movements can be determined using the signals received from accelerometers 1414. The one or more characteristics can be compared to one or more predetermined accelerometer-based thresholds at Step 1506. If the characteristics exceed one or more predetermined accelerometer-based thresholds, the characteristics can then be stored in memory of the wearable computing device 1402 at Step 1508.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step may be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A system for acquiring athletic movement data, the system comprising:
a first wearable computing device adapted to be worn on a first designated foot of a user, the first wearable computing device comprising:
one or more force-measuring sensors of the first wearable computing device;
one or more processors of the first wearable computing device coupled to the one or more force-measuring sensors of the first wearable computing device;
a memory of the first wearable computing device coupled to the one or more processors of the first wearable computing device, the memory storing instructions that, when executed by the one or more processors, cause the one or more processors to:
detect one or more signals generated by the one or more force-measuring sensors of the first wearable computing device, and determine one or more ground-reaction force values, and
in response to the one or more ground-reaction force values exceeding one or more predetermined ground-reaction force thresholds, storing the one or more ground-reaction force values in memory of the first wearable computing device.

2. The system of claim 1, wherein the one or more force-measuring sensors of the first wearable computing device comprises a piezoelectric material.

3. The system of claim 1, wherein the one or more force-measuring sensors of the first wearable computing device comprises one or more piezoresistive sensors.

4. The system of claim 1, wherein the one or more force-measuring sensors of the first wearable computing device comprises one or more force-sensing resistors.

5. The system of claim 1, wherein the one or more force-measuring sensors of the first wearable computing device comprises one or more thin-film strain gauge sensors.

6. The system of claim 1, wherein the one or more force-measuring sensors of the first wearable computing device comprises one or more thin-film capacitive sensors.

7. The system of claim 1, wherein the one or more force-measuring sensors of the first wearable computing device are at least partially disposed in an insole of a first shoe.

8. The system of claim 1, wherein the one or more force-measuring sensors of the first wearable computing device are at least partially disposed in a midsole of a first shoe.

9. The system of claim 1, wherein the one or more force-measuring sensors of the first wearable computing device are at least partially disposed on the outer surface of a first sock.

10. The system of claim 1, wherein the one or more force-measuring sensors of the first wearable computing device are disposed in an adhesive patch configured to be applied to an outer skin surface of the first designated foot, wherein the adhesive patch includes an adhesive surface and a first set of electrical contacts configured to be removably coupled to a second set of electrical contacts disposed in an insole of a first shoe.

11. The system of claim 10, wherein the second set of electrical contacts is coupled to the one or more processors of the first wearable computing device, and wherein the second set of electrical contacts, the one or more processors and the memory of the first wearable computing device are disposed in the insole of the shoe.

12. The system of claim 1, wherein the first wearable computing device further comprises a wireless communication module of the first wearable computing device coupled to the one or more processors of the first wearable computing device, and wherein the wireless communication module of the first wearable computing device is configured to transmit the stored ground-reaction force values to a local computing device.

13. The system of claim 12, wherein the wireless communication module of the first wearable computing device is further configured to transmit the stored ground-reaction force values to the local computing device according to a standard wireless networking protocol.

14. The system of claim 12, wherein the wireless communication module of the first wearable computing device is further configured to transmit the stored ground-reaction force values to the local computing device according to a Bluetooth or Bluetooth Low Energy protocol.

15. The system of claim 12, wherein the wireless communication module of the first wearable computing device is further configured to transmit the stored ground-reaction force values to the local computing device according to a Near Field Communication (NFC) protocol.

16. The system of claim 1, further comprising a local computing device, the local computing device comprising:
one or more processors of the local computing device;
a memory of the local computing device coupled to the one or more processors of the local computing device, the memory storing instructions that, when executed by the one or more processors, cause the one or more processors to extract a plurality of selected portions of the stored ground-reaction force values, the plurality of selected portions comprising an average eccentric rate of force development, an average relative concentric force, and a concentric relative impulse.

17. The system of claim 16, wherein the instructions stored in memory of the local computing device, when executed by the one or more processors of the local computing device, further cause the one or more processors to normalize the plurality of selected portions of the stored ground-reaction force values according to a database comprising a plurality of selected portions of ground-reaction force values for a population of athletes.

18. The system of claim 17, wherein the instructions to normalize the plurality of selected portions of the stored ground-reaction force values further includes instructions to calculate a T-score for each of the plurality of selected portions of the stored ground-reaction force values.

19. The system of claim 17, wherein the database comprising a plurality of selected portions of ground-reaction force values for a population of athletes resides on a remote server system, and the local computing device further comprises a network interface module configured to communicate with the remote server system.

20. The system of claim 19, wherein the remote server system comprises a cloud-based system.

21-40. (canceled)
